(19) United States
(12) Patent Application Publication
(10) Pub. No.: US 2024/0347132 A1
(43) Pub. Date: Oct. 17, 2024

(54) CLASSIFICATION OF SINGLE CELLS AS TUMOR OR NORMAL FROM SINGLE CELL SEQUENCES

(71) Applicant: Illumina, Inc., San Diego, CA (US)

(72) Inventors: Konrad Haarhoff Scheffler, Cambridge (GB); Yunjiao Zhu, San Diego, CA (US); James Han, San Carlos, CA (US); Mahdi Golkaram, San Diego, CA (US); Severine Catreux, Cardiff, CA (US); Igor Mandric, San Diego, CA (US)

(21) Appl. No.: 18/754,847

(22) Filed: Jun. 26, 2024

Related U.S. Application Data
(63) Continuation of application No. 06/262,023, filed on May 8, 1981, now Pat. No. D271793.

(51) Int. Cl. G16B 20/20; G16B 30/10

(52) U.S. Cl. CPC G16B 20/20; G16B 30/10

(57) ABSTRACT

Methods, systems, and apparatus, including computer programs encoded on computer-storage media, for classification of a single cell from a biological sample of an entity. In one aspect, the method can include obtaining data indicating a plurality of reference positions where a known variant sequence exists for the entity in respective reference positions of the plurality of reference positions, obtaining a plurality of reads for the single cell from the biological sample of the entity, determining, for respective reads of the obtained plurality of reads, a score indicating whether a variant sequence in the respective reads of the biological sample of the entity matches the plurality of reference positions where the known variant sequence exists, and classifying the single cell as a tumor cell or normal cell based on an aggregation of the score determined for the respective reads of the obtained plurality of reads.

### CROSS-REFERENCE TO RELATED APPLICATIONS

This application claims priority under 35 U.S.C. § 119 (e) to U.S. Patent Application Ser. No. 63/523,286, filed on Jun. 26, 2023, the entire contents of which is incorporated by reference in its entirety.

### BACKGROUND

Tumors can include tumor and non-tumor cells. Identification of single cells as tumor or non-tumor can be a diagnostic and therapeutic tool used in treatment of diseases such as cancer.

### SUMMARY

According to one innovative aspect of the present disclosure, a computer-implemented method for identifying one or more single cells as tumor or normal in a biological sample is disclosed. In one aspect, a method can include actions of

Other versions include corresponding systems, apparatus, and computer programs to perform the actions of methods defined by instructions encoded on computer readable storage devices. These and other versions may optionally include one or more of the following features. For instance, in some implementations, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes obtaining data indicating a plurality of reference positions where a known variant sequence exists for the entity in respective reference positions of the plurality of reference positions. The obtaining also includes obtaining, by one or more computers, a plurality of reads for the single cell from the biological sample of the entity; determining, by one or more computers and for respective reads of the obtained plurality of reads, a score indicating whether a variant sequence in the respective reads of the biological sample of the entity matches the plurality of reference positions where the known variant sequence exists. The obtaining also includes classifying, by one or more computers, the single cell as a tumor cell or normal cell based on an aggregation of the score determined for the respective reads of the obtained plurality of reads. Other embodiments of this aspect include corresponding computer systems, apparatus, and computer programs recorded on one or more computer storage devices, each configured to perform the actions of the methods.

Implementations may include one or more of the following features. The method may include: determining, by one or more computers and for respective reads of the obtained plurality of reads, a quality score corresponding to respective base calls of the respective reads corresponding to the known variant sequence, where the score indicating whether a known variant sequence of the biological sample of the entity is present in the respective reads includes the quality score. The reference sequence is a sequence that includes one or more known variant sequences in the respective reference positions or one or more known non-variant reference sequences in the respective reference positions; and the reference sequence is sequenced from a tissue sample obtained from the entity. Obtaining data indicating a plurality of reference positions includes obtaining the reference sequence. The one or more known non-variant reference sequences include sequences that do not include one or more TN somatic variants. The one or more known variant sequences in the respective reference positions include one or more TN somatic variants. The single cell from the biological sample is isolated from a non-tumor sample from the entity. The single cell from the biological sample is isolated from a tumor sample from the entity. Classifying the single cell as a tumor cell or a normal cell includes determining the following equation:

$$log \frac{P ( D \vert T )}{P ( D \vert N )} = {\sum }_{loci} [ a log a + b log b - ( a + b ) log ( a + b ) ] - {\sum }_{alt reads} log \frac{{e}_{r}}{3}$$

using the aggregation of the score determined for the respective reads of the obtained plurality of reads, where T represents a tumor cell classification, N represents a normal cell classification, D represents the score, r represents a respective read of the obtained plurality of reads, eᵣ represents the error rate of the respective read of the obtained plurality of reads r, a represents the number of respective reads that match the known variant sequence, and b represents the number of respective reads that match a known non-variant reference sequence. In some implementations, classifying the single cell as normal is based, at least in part, on the output of the equation being lower than a threshold. In some implementations, classifying the single cell as tumor is based, at least in part, on the output of the equation being lower than a threshold. The single cell is classified as tumor if the output of the equation is higher than a threshold. The reference sequence is a sequence that includes one or more known variant sequences in the respective reference positions or one or more known non-variant reference sequences in the respective reference positions; and the reference sequence is sequenced from a tissue sample. Obtaining data indicating a plurality of reference positions includes obtaining the reference sequence. The one or more known non-variant reference sequences include sequences that do not include one or more TN somatic variants. The one or more known variant sequences in the respective reference positions include one or more TN somatic variants. Implementations of the described techniques may include hardware, a method or process, or computer software on a computer-accessible medium.

One general aspect includes a method for classification of a single cell from a biological sample of an entity. The method also includes obtaining data indicating a plurality of reference positions where a known variant sequence exists for the entity in respective reference positions of the plurality of reference positions; obtaining, by one or more computers, a plurality of reads for the single cell from the biological sample of the entity; determining, by one or more computers and for respective reads of the obtained plurality of reads, a score corresponding to respective base calls of the respective reads that match the plurality of reference positions where the known variant sequence exists. The method also includes classifying, by one or more computers, the single cell as a tumor cell or normal cell based on an aggregation of the score determined for the respective reads of the obtained plurality of reads. Other embodiments of this aspect include corresponding computer systems, apparatus, and computer programs recorded on one or more computer storage devices, each configured to perform the actions of the methods.

Implementations may include one or more of the following features. The method may include: determining, by one or more computers and for respective reads of the obtained plurality of reads, a subsequent score indicating whether a variant sequence in the respective reads of the biological sample of the entity matches the plurality of reference positions where the known variant sequence exists, wherein the score corresponding to respective base calls of the respective reads includes the subsequent score. The reference sequence is a sequence that includes one or more known variant sequences in the respective reference positions or one or more known non-variant reference sequences in the respective reference positions; and the reference sequence is sequenced from a tissue sample obtained from the entity. Obtaining data indicating a plurality of reference positions includes obtaining the reference sequence. The one or more known non-variant reference sequences include sequences that do not include one or more TN somatic variants. The one or more known variant sequences in the respective reference positions include one or more TN somatic variants. The single cell from the biological sample is isolated from a non-tumor sample from the entity. The single cell from the biological sample is isolated from a tumor sample from the entity. Classifying the single cell as a tumor cell or a normal cell includes determining the following equation:

$$log \frac{P ( D \vert T )}{P ( D \vert N )} = {\sum }_{loci} [ a log a + b log b - ( a + b ) log ( a + b ) ] - {\sum }_{alt reads} log \frac{{e}_{r}}{3}$$

using the aggregation of the score determined for the respective reads of the obtained plurality of reads, where T represents a tumor cell classification, N represents a normal cell classification, D represents the score, r represents a respective read of the obtained plurality of reads, eᵣ represents the error rate of the respective read of the obtained plurality of reads r, a represents the number of respective reads that match the known variant sequence, and b represents the number of respective reads that match a known non-variant reference sequence. In some implementations, classifying the single cell as normal is based, at least in part, on the output of the equation being lower than a threshold. In some implementations, classifying the single cell as tumor is based, at least in part, on the output of the equation being lower than a threshold. Implementations of the described techniques may include hardware, a method or process, or computer software on a computer-accessible medium.

One general aspect includes a method for classification of a single cell from a biological sample of an entity. The method also includes obtaining data indicating a plurality of reference positions where a known variant sequence exists for the entity in respective reference positions of the plurality of reference positions; obtaining, by one or more computers, a plurality of reads for the single cell from the biological sample of the entity; determining, by one or more computers and for respective reads of the obtained plurality of reads, a first score indicating whether a variant sequence in the respective reads of the biological sample of the entity matches the plurality of reference positions where the known variant sequence exists; determining, by one or more computers and for respective reads of the obtained plurality of reads, a second score corresponding to respective base calls of the respective reads that match the plurality of reference positions where the known variant sequence exists. The method also includes classifying, by one or more computers, the single cell as a tumor cell or normal cell based on an aggregation of the first score and the second score determined for the respective reads of the obtained plurality of reads. Other embodiments of this aspect include corresponding computer systems, apparatus, and computer programs recorded on one or more computer storage devices, each configured to perform the actions of the methods.

Implementations may include one or more of the following features. In some implementations, the reference sequence is a sequence that includes one or more known variant sequences in the respective reference positions or one or more known non-variant reference sequences in the respective reference positions; and the reference sequence is sequenced from a tissue sample obtained from the entity. In some embodiments, obtaining data indicating a plurality of reference positions includes obtaining the reference sequence. In some embodiments, the one or more known non-variant reference sequences include sequences that do not include one or more TN somatic variants. The one or more known variant sequences in the respective reference positions include one or more TN somatic variants. In some example embodiments, the single cell from the biological sample is isolated from a non-tumor sample from the entity. In other example embodiments, the single cell from the biological sample is isolated from a tumor sample from the entity. Classifying the single cell as a tumor cell or a normal cell includes determining the following equation:

$$log \frac{P ( D \vert T )}{P ( D \vert N )} = {\sum }_{loci} [ a log a + b log b - ( a + b ) log ( a + b ) ] - {\sum }_{alt reads} log \frac{{e}_{r}}{3}$$

using the aggregation of the first score and the second score determined for the respective reads of the obtained plurality of reads, where T represents a tumor cell classification, N represents a normal cell classification, D represents the first score and the second score, r represents a respective read of the obtained plurality of reads, eᵣ represents the error rate of the respective read of the obtained plurality of reads r, a represents the number of respective reads that match the known variant sequence, and b represents the number of respective reads that match a known non-variant reference sequence. In some implementations, classifying the single cell as normal is based, at least in part, on the output of the equation being lower than a threshold. In some implementations, classifying the single cell as tumor is based, at least in part, on the output of the equation being lower than a threshold. Implementations of the described techniques may include hardware, a method or process, or computer software on a computer-accessible medium.

These and other innovative aspects of the present disclosure are readily apparent in view of the detailed description, the accompanying drawings, and the claims.

### BRIEF DESCRIPTION OF THE DRAWINGS

FIG. 1 is a block diagram of an example of a system for classification of single cells as tumor or normal from single cell sequences.

FIG. 2 is a flowchart of an example of a process for performing classification of single cells as tumor or normal from single cell sequences.

FIG. 3 is another flowchart of an example of a process for performing classification of single cells as tumor or normal from single cell sequences.

FIG. 4 is another flowchart of an example of a process for performing classification of single cells as tumor or normal from single cell sequences.

FIG. 5 is a block diagram of system components that can be used to implement a system for classification of single cells as tumor or normal from single cell sequences.

### DETAILED DESCRIPTION

The present disclosure is directed to systems, methods, apparatuses, computer programs, or any combination thereof, for classification of single cells as tumor or normal based on single cell sequence reads. Tumor cells are known to exhibit genetic, epigenetic, and phenotypic heterogeneity. The accurate identification of a single cell as tumor or normal can be important to the identification of disease, research, and treatment selection. For example, accurate identification of an individual cell as tumor or normal can be important to the understanding of tumor heterogeneity. Accurate identification at the single-cell level can provide a more complete understanding of tumor heterogeneity, enabling researchers and health care providers to identify different subpopulations of cells with distinct properties, such as drug resistance or metastatic potential. Accordingly, the accurate classification of single cells can guide treatment decisions for a subject affected by tumors (e.g., malignant or benign) and increase the understanding of genetic, epigenetic, and phenotypic diversity of tumor cells within a tumor or across different tumors.

In order to accurately identify single cells as tumor or normal, tens of thousands of reads can be analyzed for a respective single cell. In some example embodiments, the analysis of each respective read (tens of thousands per single cell) can include determining one or more scores for each of the respective tens of thousands of reads. In this implementation, the aggregate of the determined scores for each of the respective tens of thousands of reads can classify a single cell as tumor or normal. In some example embodiments, the score can be based on one or more variables that are used to determine a classification of the single cell as tumor or normal. For example, the variables can include a likelihood that a respective read includes one or more variant sequences (e.g., a single nucleotide variant (SNV) also called a TN somatic variant an alteration in gene expression) and/or a base call quality score corresponding to each base call of the respective read.

In some example embodiments, the classification of a single cell as tumor or normal can include an aggregate of more than one scored variable determined of each of the respective tens of thousands of reads. For example, first, the respective (e.g., tens of thousands) reads for the single cell are scored using a first score to indicate a likelihood the read includes one or more variant sequences (e.g., SNV or alterations in gene expression). Second, the respective reads are scored using a second score that is based on the base call quality score corresponding to each base call of the respective read. The present disclosure then classifies the single cell as a normal cell or a tumor cell based on the aggregated first score and second score determined for the respective reads of the tens of thousands of reads.

The classification of a single cell as a tumor cell or normal cells is a technological improvement in the field of biological classification. The accurate identification of a single cell as tumor or normal can be important to the identification of disease, research, and treatment selection. For example, accurate identification of an individual cell as tumor or normal can be important to the understanding of tumor heterogeneity. Tumor cells are known to exhibit genetic, epigenetic, and phenotypic heterogeneity. Accurate identification at the single-cell level can provide a more complete understanding of tumor heterogeneity, enabling researchers and health care providers to identify different subpopulations of cells with distinct properties, such as drug resistance or metastatic potential.

Prior methods to classify biological samples as tumor or normal at the granularity of a single cell have failed. However, the techniques of the present disclosure solve this problem and enable advances in the evaluation of, e.g., the effectiveness of a prior cancer treatment for an individual. That is, given the knowledge of a known variant sequence of a particular entity's cancer, biological samples can be obtained from the entity at predetermined intervals after cancer removal or treatment and heterogeneity of normal vs. tumor cells in the biological sample can be evaluated, using the techniques of the present disclosure, to determine whether the cancer is recurring. The present disclosure improves the performance of this downstream analysis of the cell classification.

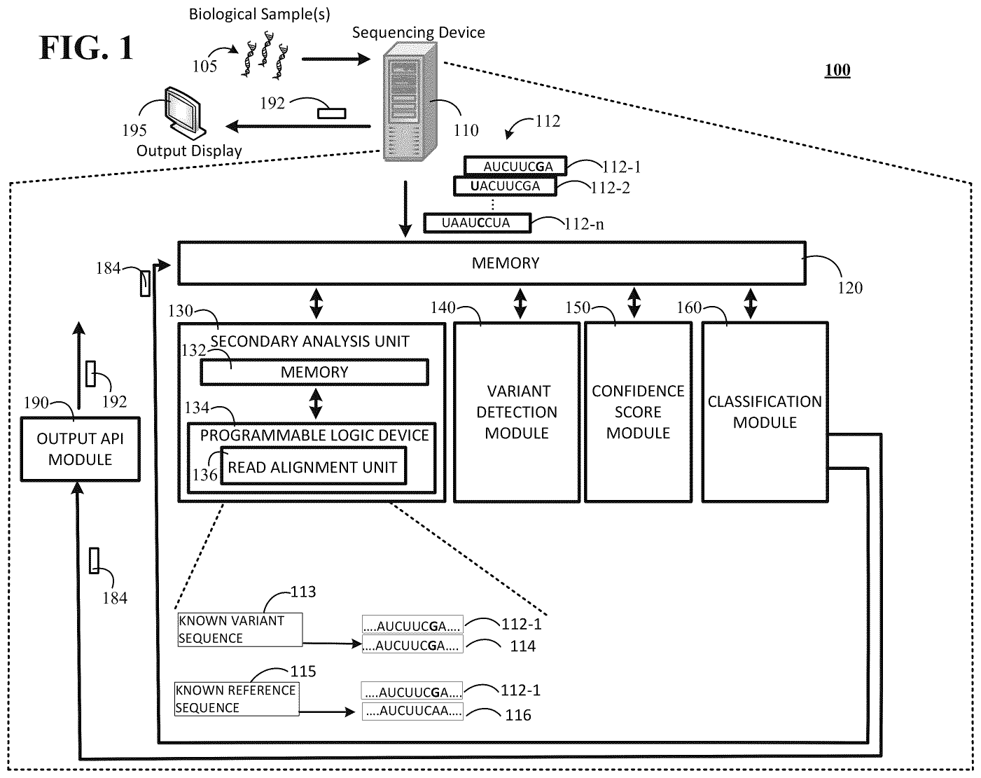

FIG. 1 is a block diagram of an example of a system 100 for classification of a single cell as tumor or normal from single cell sequence reads. The system 100 can include a nucleotide sequencing device 110, a memory 120, a secondary analysis unit 130, variant detection engine 140, confidence score engine 150, and a classification engine 160, an output application program interface (API) engine 190, and an output display 195. In the example of FIG. 1, each of these components is described as being implemented within the nucleic acid sequencing device 110. However, the present disclosure is not limited to such embodiments.

Instead, in some implementations, one or more of the “units” or “engines” described in FIG. 1 can be executed on a computer outside the nucleic acid sequencing device 110. For example, in some implementations, the secondary analysis unit 130 may be implemented within the nucleic acid sequencing device 110 and the variant detection engine 140, a confidence score engine 150, a classification engine 160, an output application program interface (API) engine 190 can be implemented in one or more different computers outside of the sequencing device 110. In such implementations, the one or more different computers and the nucleic acid sequencing device 110 can be communicatively coupled using one or more wired networks, one or more wireless networks, or a combination thereof. In such implementations, for example, the network may be one or more of a wired Ethernet, a wired optical network, a LAN, a WAN, a cellular network, the Internet, or a combination thereof. While, in some implementations, one or more of the computers communicatively coupled to the nucleic acid sequencing device 110 can be a remote cloud server, the present disclosure is not so limited. Instead, in other implementations, the one or more computers can connected to the sequencing device 110 via a direct connection such as a direct Ethernet connection, a USB-C connection, or the like.

For purposes of this specification, the term “engine” includes one or more software components, one or more hardware components, or any combination thereof, which can be used to realize the functionality attributed to a respective engine by this specification. In general, an “engine,” as described herein, uses one or more processors to execute software instructions to realize the functionality of the engine described herein. A processor can include a central processing unit (CPU), graphics processing unit (GPU), or the like.

Likewise, the term “unit” as used in this specification includes one or more software components, one or more hardware components, or any combination thereof, which can be used to realize the functionality attributed to a respective unit by this specification. In general, a “unit,” as described herein, uses one or more hardware components such as hardwired digital logic gates or hardwired digital logic blocks arranged as processing engines to perform operations that realize the functionality of the unit described herein. Such hardwired digital logic gates or hardwired digital logic circuits can include a field programmable gate array (FPGA), an application specific integrated circuit (ASIC), or the like.

The nucleic acid sequencing device 110 (also referred to herein as sequencing device 110) is configured to perform primary nucleic acid sequence analysis. In particular, the sequencing device 110 is configured to perform single cell sequencing. In such implementations, the biological sample 105 sequenced by the sequencing device 110 can be comprised of a single cell.

In some embodiments, the single cell is isolated from tissue. Non-limiting examples of tissue include whole blood, peripheral blood mononuclear cells (PBMCs), saliva, tumor tissue, non-tumor tissue, urine, sweat, cerebral spinal fluid, etc. In some embodiments, individual cells can be isolated from a tissue sample using a variety of techniques, such as fluorescence-activated cell sorting (FACS), micromanipulation, or laser capture microdissection. In this example isolated cells are then lysed to release their DNA or RNA, which is amplified using various methods to generate sufficient material for sequencing. Different amplification methods can be used depending on whether DNA or RNA is being sequenced. In this example, once the DNA or RNA has been amplified, it can be prepared for sequencing using a library preparation method that adds adapter sequences to the ends of the amplified fragments. These adapters allow the fragments to be attached to a sequencing flow cell and amplified further using bridge amplification or clonal amplification methods.

The sequencing device 110 is configured to generate ordered sequences of nucleotides, respectively referred to herein as “reads” or “sequence reads.” In particular, in the implementation of FIG. 1, the nucleic acid sequencer 110 can be used to produce RNA reads of a biological sample 105. In such implementations, this can occur using RNA-seq protocols. By way of example, a biological sample can be preprocessed using reverse-transcription to form complementary DNA (cDNA) using a reverse transcriptase enzyme. In other implementations, the nucleic acid sequencer 110 can include an RNA sequencer, and the biological sample 105 can include an RNA sample. RNA reads produced using cDNA or via an RNA sequencer can be comprised of C, G, A, and Uracil (U). However, though implementations of the present disclosure are described with respect to RNA sequences, the same operations can be performed on DNA reads generated by the nucleic acid sequencer without the reverse-transcription operations described above to produce cDNA.

With reference to the example of FIG. 1, the sequencing device 110 can sequence the biological sample 105 (e.g., a single cell) and generate a corresponding set of RNA reads (e.g., tens of thousands of reads) represented using base calls corresponding to nucleotides of A, C, U, and G. In this example, the RNA sequence reads 112-1, 112-2, 112-n are output by the sequencing device 110 and stored in the memory device 120. The memory device 120 can be accessible by each of the components of FIG. 1 including the secondary analysis unit 130, variant detection engine 140, confidence score engine 150, the classification engine 160, and the output API engine 190. Though respective engines may be depicted as providing an output of a first engine to a second engine, practical implementation of such a feature may include the first engine storing the output in a memory device such as memory 120 and the second engine accessing the stored output from the memory device and processing the accessed output as an input to the second engine.

The secondary analysis unit 130 can access the reads 112-1, 112-2, 112-n stored in the memory device 120 and perform one or more secondary analysis operations on the reads 112-1, 112-2, 112-n. In some implementations, the reads 112-1, 112-2, 112-n may be stored in the memory device 120 in compressed data records. In such implementations, the secondary analysis unit 130 can perform decompression operations on the compressed read records prior to performing secondary analysis operations on the read records. Secondary analysis operations can include mapping one or more reads to a reference sequence stored in memory device 120, aligning one or more reads to the reference sequence, or both. In addition to performance of secondary analysis operations, the secondary analysis unit 130 can also be configured to perform sorting operations. Sorting operations can include, for example, ordering reads that have been aligned by the secondary analysis unit 130 based on the position in the reference genome to which the aligned reads were mapped.

The functionality of the read alignment unit 136 can include obtaining data indicating a plurality of reference positions where a known variant sequence exists in respective reference positions of the plurality of reference positions. For example, obtaining data indicating a plurality of reference positions can include obtaining a reference sequence. A reference sequence includes a sequence (e.g., nucleic acid, amino acid, peptide, or chromosome) that has known characteristics and can serve as a template for comparisons with other sequences. For example, a reference sequence can be a high-quality, annotated, and well-characterized sequence that represents the consensus sequence of a particular species, organism, or biological sample. In some embodiments, a reference sequence can provide a framework for the study of genetic variation, gene expression, and functional genomics. For example, a reference sequence can be used as a basis for comparing and analyzing genetic variations in different populations, individuals, or tissues from individuals.

In some example embodiments, a reference sequence is a sequence that includes one or more known variant sequences in the respective reference positions. In some example embodiments, a reference sequence is a sequence that includes one or more known non-variant reference sequences in the respective reference positions. In some example embodiments, a reference sequence is a sequence that includes one or more known variant sequences in respective reference positions and one or more known non-variant reference sequences in the respective reference positions.

The functionality of the read alignment unit 136 can also include obtaining one or more reads such as RNA reads 112-1, 112-2, 112-n that were stored in memory 120 by the sequencing device 110, mapping the obtained reads 112-1, 112-2, 112-n to one or more reference sequence locations of a reference sequence, and then aligning the mapped reads 112-1, 112-2, 112-n to the reference sequence.

In the example of FIG. 1 sequence reads 112-1, 112-2, 112-n are compared to a known reference sequence using read alignment unit 136. Here, in this example, the reference sequence is a sequence generated by sequencing an initial tissue sample of the same entity from which the single cell biological sample 105 was obtained. In some implementations, the initial tissue sample may be a tumor that formed a portion of the entity's body (e.g., lung, pancreas, stomach, etc.) and the single cell may be obtained from the same portion of the entity's body on which the tumor formed.

In some implementations, the single cell may be obtained from a new tumor that has formed after the tumor (which yielded the initial tissue sample) has been removed. Since tissue samples such as tumor tissue samples can comprise both tumor cells and normal cells, the reference sequence in this implementation was analyzed to identify normal sequences (e.g., known reference sequence 115) and tumor supporting sequences (known variant sequences 113). For example, a non-single cell biological sample from the tissue of an entity can be sequenced to perform tumor normal (SNV calling). This process can identify variants that are present in tumor samples but not present in non-tumor samples. In this example, the sequencing method can be whole genome sequencing (WGS) or whole exome sequencing (WES), or any technology that generates a fingerprint of tumor specific SNVs (also called a TN somatic variant). In other embodiments, the reference sequence can include a known tumor genomic library with a plurality of known variant sequences or a known tumor gene expression library with a plurality of known variant sequences. Given the known reference sequence of an entity having, e.g., a tumor with a known variant sequence, single-cell reads 112 generated based on a cell obtained from a subsequent sample can be analyzed in view of the known reference sequence using the techniques described herein.

In this implementation, the secondary analysis unit 130 can access the known reference sequence 115, the known variant sequence 113, or both, stored in the memory device 120 and perform one or more secondary analysis operations on the reads the known reference sequence 115, the known variant sequence 113, or both. In some implementations, the known reference sequence 115, the known variant sequence 113, or both, may be stored in the memory device 120 in compressed data records. In such implementations, the secondary analysis unit 130 can perform decompression operations on the compressed read records prior to performing secondary analysis operations on the read records.

In some implementations, the known variant sequence 113 can include a combination of TN somatic variants. For example, a single TN somatic variant or a combination of TN somatic variants in the known variant sequence 113 can be indicative of a particular tumor or biological sample. In this example, the obtained reads 112-1, 112-2, 112-n can be mapped by the read alignment unit 136 to the known reference sequence such as known variant sequence 113. However, in some embodiments, the reference sequence such as reference sequence 115 does not include the TN somatic variants. In such instances, the read alignment unit 136 can align reads that match the known reference sequence when the reads do not contain a TN somatic mutation.

With reference to FIG. 1, the read alignment unit can align read 112-1 with the reference sequence 113. In this example, an eight base call portion 114 of the known variant sequence 113 is shown with the sequence AUCUUCGA which represents a TN somatic variant. The read 112-1 is aligned with the known variant sequence 113 because nucleotide portion 114 of the known variant sequence 113 matches the read 112-1. In this example, an eight nucleotide portion 116 of the known reference sequence 115 is shown with the sequence AUCUUCAA. The read 112-1 is not aligned with the known variant sequence 115 because nucleotide portion 116 of the known reference sequence 115 does not match. Read records describing the aligned reads can be output by the secondary analysis unit 130 and stored in the memory for later access by one or more other engines of system 100 such as the variant detection engine 140. In some implementations, a read record can be stored for each single-cell read 112 indicating whether or not the single cell read such as 112-1 includes a known variant sequence.

In some examples, the reference sequence can be autogenous. For example, the single cell biological sample 105 from which the sequencing device 110 generates reads 112-1, 112-2, 112-n is a single cell that was isolated from the same biological sample from which the reference sequence was obtained. In some embodiments, the single cell biological sample 105 from which the sequencing device 110 generates reads 112-1, 112-2, 112-n is a single cell that was isolated from a biological sample that was adjacent to a biological sample from which the reference sequence was obtained. For example, the single cell biological sample 105 could be isolated from tissue that is adjacent to a location where a tumor was removed from the entity. In this case, the reference sequence could be generated from the tissue of the removed tumor. In some embodiments, the single cell biological sample 105 from which the sequencing device 110 generates reads 112-1, 112-2, 112-n is a single cell that was isolated from a metastatic tumor. For example, the single cell biological sample 105 could be isolated from tumor tissue that has metastasized from an initial tumor. In this case, the reference sequence could be generated from the initial tumor.

Execution of the system 100 can begin with the sequencing device 110 sequencing the biological sample 105 (e.g., a single cell). Sequencing the biological sample 105 can include generating, by the sequencing device 110, read sequences 112-1, 112-2, and 112-n that are a data representation of the ordered sequences of nucleotides present in the biological sample 105, wherein n is any integer larger than 1. For example, a single cell biological sample 105 may generate tens of thousands of reads 112. For example, about 10³ to about 10⁶ reads can be generated from a single cell. In some embodiments, the system 100 is configured to sequence RNA reads, using techniques described above, and the reads generated by the sequencing device 110 can be stored in the memory 120.

The variant detection engine 140 can obtain read records corresponding to a batch of aligned and sorted reads that were aligned by the read alignment unit 136 and determine if each read records corresponds to a single cell read sequence that includes a known variant sequence. In some implementations, this can be achieved by determining whether the obtained read record corresponds to a read such as 112-1 that aligns with the known variant sequence 113 or the known reference sequence 115. In this example, the variant detection engine 140 would determine that the read 112-1 includes a variant sequence (e.g., a TN somatic mutation). However, the same result can be determined in different ways. For example, in some implementations, the variant detection engine 140 may determine that read 112-1 does not align with the known, normal reference sequence 115 by analyzing the nucleic acids 116 compared to the nucleic acids of the read 112-1. In such instances, if the read 112-1 does not match the known, normal reference sequence, then the variant detection engine 140 may determine that the read 112-1 includes a variant signature, as the different base calls forming the variant signature is the reason the read 112-1 did not match the known, normal reference sequence.

The variant detection engine 140 can determine a first score, for each of the reads (e.g., the respective reads) 112, based on the alignment of each of the reads 112 with the reference sequence. In some embodiments, the first score associated with each read may be, e.g., a “1” or “0” based on whether the variant detection engine determines, that the particular read, includes a known variant sequence. In such an implementation, a “1” associated with a read can indicate that the read includes a known variant sequence and a “0” associated with a read can indicate that the read does not include a known variant sequence. While the example of a “1” and “O” is provided, other scores or metadata can be associated with a read to indicate whether or not the read includes a known variant sequence. In some implementations, the variant detection engine 140 relies on data within a read record produced by the alignment unit 136 indicating whether a read such as read 112-1 matches a known variant sequence 113 or a known reference sequence 115. In other implementations, the variant detection engine 140 can perform a comparison of a read such as read 112-1 to make the determination as to whether read matches a known variant sequence 113 or a known reference sequence 115. Regardless of implementation, the variant detection engine 140 can generate output data indicating a first score for each single cell read 112, whether the read includes a known variant sequence.

The confidence score engine 150 is configured to generate a second score for each read that provides an indication of the level of quality of each base call of the read being scored. The second score can be based on a base quality score of each base call of the single sequence read such as read 112-1 that corresponds to a known variant sequence. The base quality score is generated by nucleic acid sequencer for each base of a read as an indication of the level of confidence that the sequencer 110 called the correct base at each respective location of the read. Thus, a high base quality score indicates that there is a low likelihood of potential sequencing errors or artifacts in a read. Alternatively, a low base quality score indicates that there is a high likelihood of a potential sequencing errors or artifacts in a read.

The second score based on the base quality score thus adds a quality score component to the analysis of whether a single-cell read such as 112-1 includes a known variant sequence. This is informative as a read determined by the variant detection engine 140 as including a known variant sequence may, in fact, be a false positive if one or more of the bases in the read corresponding to the known variant signature have low base quality scores. Such low base quality scores may indicate that the read only appears to have the known variant signature because one or more bases were erroneously called during sequencing. On the other hand, a determination by the variant detection engine 140 that a single-cell read includes a known variant sequence can be affirmed by high base quality scores at each based of a single-cell read corresponding to a known variant sequence.

In some implementations, a base quality score may be, e.g., a Phred quality score. The Phred quality score is a logarithmic measure of the probability that the base call is incorrect. The Phred score is calculated as: Q=−10*log 10(P), where Q is the quality score and P is the probability of an error. For example, a base call with a Phred score of 20 indicates a 1 in 100 chance that the base call is incorrect. The probability of an error is determined by comparing the observed signal intensity at a given position to the expected signal intensity based on the sequencing platform's error rates and noise characteristics. In addition, the quality score may be influenced by other factors, such as the quality of the raw sequencing data, the complexity of the RNA sequence, and the alignment of the sequence to a reference sequence.

In some implementations, the second score may be generated based on base quality scores for only those base calls of a single-cell read such as read 112-1 that corresponds to a known variant sequence. However, the present disclosure is not so limited. Instead, the second score (e.g., the base call quality score) can be applied to any number of nucleotides in a read. In some embodiments, for example, the second score for a single-cell read can be determined based on the base quality score for each base call of the single-cell read. Thus, the confidence score engine 150 assigns a second score to each single-cell read such as read 112-1 based on a base call quality score of one or more base calls of the read 112-1.

The classification engine 160 is configured to determine, based on an aggregation of the first score and the second score for each of the plurality of single-cell reads, a classification of the single cell as a tumor cell or a normal cell. For example, the classification engine 160 can receive as an input, multiple different parameters. These parameters, as will be discussed in more detail below, include a number of alt-supporting reads, a number of ref-supporting reads, and a base call error rate. The value of each of these parameters, for each single-cell read, can be determined based on the first score and the second score.

For example, the classification engine 160 can use the first score to provide an indication of (i) a number of single-cell reads that support a known variant sequence 113 and (ii) a number of single-cell reads that support a known reference sequence 115. By way of example, the number of single-cell reads supporting a known variant sequence can be a sum of the number of single-cell reads that have a “1” as their first score and the number of single-cell reads supporting a known reference can be a sum of the number of reads having a “0” as their first score. These values can be used as input to the classification algorithm. Likewise, the classification engine 160 can determine a base call error rate based on the second score for each single-cell read. For example, the classification engine can determine that any single-cell read having a second score that satisfies a predetermined threshold has a sufficient base call quality and those below it have insufficient base call quality. Then, the base call error rate can be determined as a ratio of the single-cell reads having, e.g., insufficient base call quality over the total number of single-cell reads.

In more detail, in the classification algorithm below, the following notation is used: r: read, f: alt allele frequency, e: base call error rate as obtained from base call quality score, a: number of alt-supporting reads (i.e., the number of reads that were determined to align with a portion e.g., 114 of the known variant sequence 113), and b: number of ref-supporting reads (i.e., a number of reads that matched the known reference sequence 115 (e.g., a known non-variant reference sequence) by aligning to a portion e.g., portion 116 of the known reference sequence 115). A maximum likelihood approach can be used to approximate a Bayesian solution. For example, the alt allele frequency is estimated as if an allele frequency is directly observable from the reads 112,

$$f = \frac{a}{a + b} \qquad ( 1 )$$

where a is the number of alt-supporting reads and b is the number of ref-supporting reads at the locus. In some cases, this can be inaccurate at low coverage but converges to the correct solution as coverage increases. One property of the maximum likelihood approach is that it does not consider a locus to provide evidence in favor of the normal hypothesis, because even a locus with no alt-supporting reads is treated as supporting both hypotheses. Instead of Equation 1 we then have (at any one locus):

$$P ( R \vert T ) = {\prod }_{r} P ( r \vert f ) \qquad ( 2 )$$

With equation 1 for the tumor hypothesis, f=0 for the normal hypothesis, and P(r|f) is defined by:

$$P ( r \vert f ) = \{ \begin{matrix}f ( 1 - {e}_{r} ) + ( 1 - f ) \frac{{e}_{r}}{3} for alt reads \\ ( 1 - f ) ( 1 - {e}_{r} ) + f \frac{{e}_{r}}{3} for ref reads\end{matrix} \qquad ( 3 )$$

It is assumed that:

$$e ≪ 1 , \qquad ( 4 )$$

$$\frac{e}{3} ≪ \frac{a}{b} ,$$

$$and$$

$$\frac{e}{3} ≪ \frac{b}{a} ,$$

The assumption of operation (4) allows the calculation of the overall log-likelihood difference as follows (treating 0 log 0 as equal to 0 because it is really shorthand for 0 log ε where ε is a small positive value):

$$log \frac{P ( D \vert T )}{P ( D \vert N )} = {\sum }_{loci} [ a log a + b log b - ( a + b ) log ( a + b ) ] - {\sum }_{alt reads} log \frac{{e}_{r}}{3} \qquad ( 5 )$$

Said differently, in some implementations, equation (5) can be used to calculate the likelihood ratio between two hypotheses (T and N) based on data (D) obtained from sequencing reads. The data (D) can be the first rule (e.g., the first score) and/or the second rule (e.g., the second score). In such implementations, equation (5) can compare the probability of observing the (D) under each hypothesis, given the values of the parameters that describe the variation at each location in the (sequence). The left-hand side

$$( log \frac{P ( D \vert T )}{P ( D \vert N )} )$$

of the equation calculates the Bayes factor and measures the relative strength of evidence in favor of one hypothesis over the other. For example, the logarithm of the ratio of the probability of observing the data (D) under the two hypotheses (T and N). The right-hand side of the equation is a sum over all loci in the sequence and depends on the number of alt alleles (a) and ref alleles (b) at each locus, as well as their frequencies (f) (e.g., the first rule). In this way, certain locations in the sequence can be weighted to be more informative than others, depending on the nature and frequency of the variant. The second part of the equation calculates the contribution of each read to the Bayes factor. It is a sum over all alt-supporting reads at each locus and takes into account the error rate (e) of the base calls obtained from the base call quality score (i.e., the second rule). The error rate (e) reflects the fact that sequencing errors can introduce noise and reduce the reliability of the data.

The classification engine 160 can generate a likelihood, based upon execution of equation (5) above, that a cell is a tumor cell or a normal cell. In some implementations, the classification engine 160 can determine whether the generated likelihood satisfies a predetermined threshold. If the classification engine 160 determines that the generated likelihood satisfies the predetermined threshold, then the classification engine can generate output data 184 indicating that the single cell is a tumor cell. Alternatively, if the classification engine 160 determines that the generated likelihood does not satisfy the predetermined threshold, the classification engine 160 can generate output data 184 indicating that the single cell is a normal cell. The classification engine 160 can generate output data 184 based on the comparison of the generated likelihood to the predetermined threshold, with the output data 184 including data indicating a classification of the single cell as tumor or normal.

In some implementations, the data indicating the classification of the single cell as tumor or normal in the output data 184 can include a binary classification of the single cell as tumor or normal. In some implementations, this output data 182 can be stored in the memory 120 for subsequent use by another computing engine, for subsequent output to a user device, or the like.

Alternatively, or in addition, the classification engine 160 can generate output data 184 that can be provided as an input to the output application programming interface (API) engine 190. In such instances, the output data 184 can include rendering data that, when rendered by the API engine, causes an output display to output indicating whether each of the single cell sequenced by the sequencing device 110 is classified as tumor or normal. This can include causing the output display 195 to display any of the output data 184 stored in the memory 120 associated with the analyzed single cell. In some implementations, this output can be displayed in the form of a report.

Other types of output 192 can be provided by the output API engine 190. For example, in some implementations, the output 192 can be data that causes another device such as a printer to output a report that includes data identifying the each of the single cells sequenced by the sequencing device 110 is classified as tumor or normal. In other implementations, this output data 192 can cause a speaker to output audio data that includes each of the single cells sequenced by the sequencing device 110 is classified as tumor or normal. Other types of output data can also be triggered by the output API engines 190.

In some implementations, the output display 195 can be a display panel of the sequencing device 110. In other implementations, the output display 195 can be a display panel of a user device that is connected to the sequencing device 110 using one or more networks. Indeed, the sequencing device 110 can be used to communicate the output data 192 to any device having any display.

The accurate classification of single cells as tumor or normal as described herein can provide multiple technological advantages. For example, the accurate classification of single cells as tumor or normal can be advantageous to the field of personalized medicine and provide insights into the genetic and molecular characteristics of individual tumors, which can be used to develop personalized cancer treatments. For example, specific genetic mutations or alterations in gene expression may make certain cells more susceptible to particular therapies.

In some instances, accurate identification of a single cell as tumor or normal as disclosed herein can inform researchers and health care providers if a newly identified tumor is the same or has similar genetic characteristics as a tumor that has been previously treated (e.g., removed from the subject). For example, accurate identification of tumor cells at the single-cell level can help to monitor treatment response and assess the effectiveness of cancer therapies. This can enable clinicians to modify treatment regimens in real-time to optimize patient outcomes.

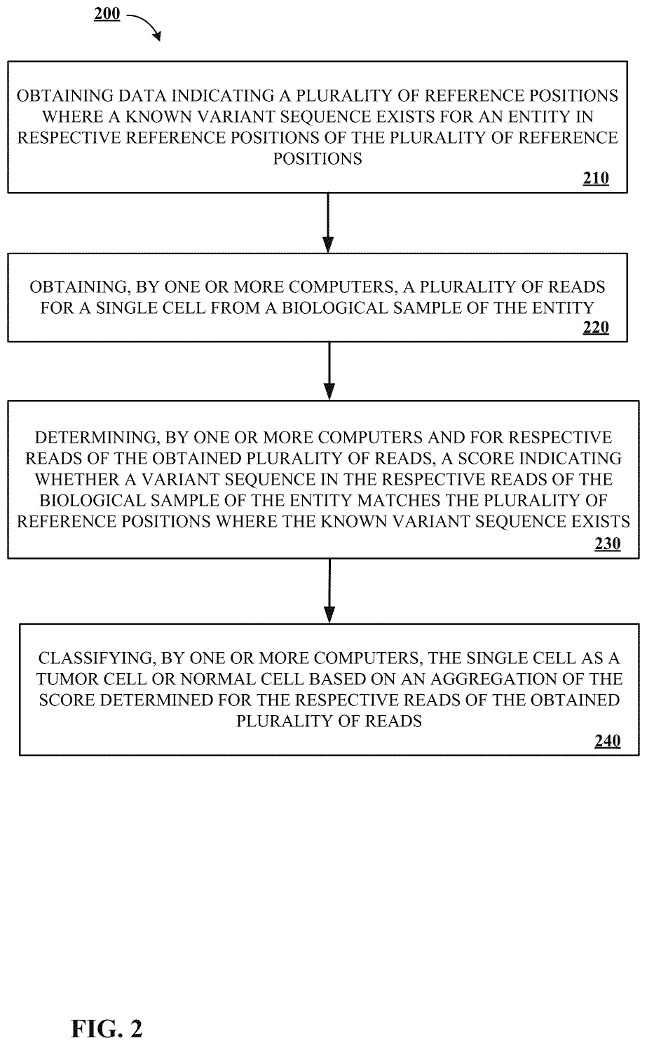

FIG. 2 is a flowchart of an example of a process 200 for performing classification of single cells as tumor or normal from single cell sequences. The process 200 may be performed by one or more electronic systems, for example, the system 100 of FIG. 1.

The process 200 includes obtaining data indicating a plurality of reference positions where a known variant sequence exists for an entity in respective reference positions of the plurality of reference positions (210). For example, functionality of the read alignment unit 136 obtaining data indicating a plurality of reference positions can include obtaining a reference sequence. In some example embodiments, a reference sequence is a sequence that includes one or more known non-variant reference sequences in the respective reference positions. In some example embodiments, a reference sequence is a sequence that includes one or more known variant sequences in respective reference positions and one or more known non-variant reference sequences in the respective reference positions. In some examples, obtaining data indicating a plurality of reference positions includes obtaining the reference sequence.

In certain embodiments, the process of WGS can be used to obtain a known variant sequence of an entity. For example, WGS can be performed to sequence the complete genome of an entity, such as a human. Subsequently, the obtained genome sequence can be aligned and compared to a reference sequence, such as a reference human genome (e.g., a non-variant sequence). By comparing the two sequences, any variations in the WGS data can be identified. Through this approach, the whole genome sequence obtained from the entity's WGS can be utilized as the known variant sequence to classify single cells from the entity as tumor or normal.

The process 200 includes obtaining, by one or more computers, a plurality of reads for a single cell from a biological sample of the entity (220). For example, the functionality of the read alignment unit 136 can also include obtaining one or more reads such as RNA reads 112-1, 112-2, 112-n that were stored in memory 120 by the sequencing device 110, mapping the obtained reads 112-1, 112-2, 112-n to one or more reference sequence locations of a reference sequence, and then aligning the mapped reads 112-1, 112-2, 112-n to the reference sequence.

The process 200 includes determining, by one or more computers and for respective reads of the obtained plurality of reads, a score indicating whether a variant sequence in the respective reads of the biological sample of the entity matches the plurality of reference positions where the known variant sequence exists (230). For example, the classification engine 160 can use the score to provide an indication of (i) a number of single-cell reads that support a known variant sequence 113 and (ii) a number of single-cell reads that support a known reference sequence 115. By way of example, the number of single-cell reads supporting a known variant sequence can be a sum of the number of single-cell reads that have a “1” as their score and the number of single-cell reads supporting a known reference can be a sum of the number of reads having a “0” as their score. These values can be used as input to the classification algorithm.

The process 200 includes classifying, by one or more computers, the single cell as a tumor cell or normal cell based on an aggregation of the score determined for the respective reads of the obtained plurality of reads (240). For example, the classification engine 160 can generate a likelihood, based upon execution of equation (5) above, that a cell is a tumor cell or a normal cell. In some implementations, the classification engine 160 can determine whether the generated likelihood satisfies a predetermined threshold. If the classification engine 160 determines that the generated likelihood satisfies the predetermined threshold, then the classification engine can generate output data 184 indicating that the single cell is a tumor cell. Alternatively, if the classification engine 160 determines that the generated likelihood does not satisfy the predetermined threshold, the classification engine 160 can generate output data 184 indicating that the single cell is a normal cell.

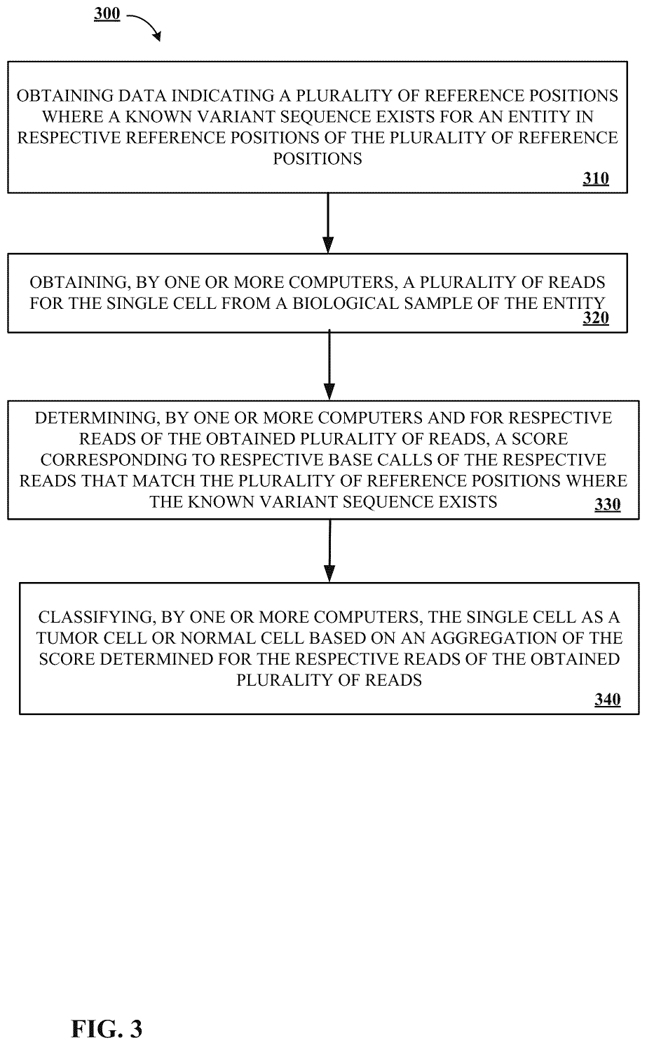

FIG. 3 is a flowchart of an example of a process 300 for performing classification of single cells as tumor or normal from single cell sequences. The process 300 may be performed by one or more electronic systems, for example, the system 100 of FIG. 1.

The process 300 includes obtaining data indicating a plurality of reference positions where a known variant sequence exists for an entity in respective reference positions of the plurality of reference positions (310). For example, functionality of the read alignment unit 136 obtaining data indicating a plurality of reference positions can include obtaining a reference sequence. In some example embodiments, a reference sequence is a sequence that includes one or more known non-variant reference sequences in the respective reference positions. In some example embodiments, a reference sequence is a sequence that includes one or more known variant sequences in respective reference positions and one or more known non-variant reference sequences in the respective reference positions. In some example, obtaining data indicating a plurality of reference positions includes obtaining the reference sequence.

The process 300 includes obtaining, by one or more computers, a plurality of reads for the single cell from a biological sample of the entity (320). For example, the functionality of the read alignment unit 136 can also include obtaining one or more reads such as RNA reads 112-1, 112-2, 112-n that were stored in memory 120 by the sequencing device 110, mapping the obtained reads 112-1, 112-2, 112-n to one or more reference sequence locations of a reference sequence, and then aligning the mapped reads 112-1, 112-2, 112-n to the reference sequence.

The process 300 includes determining, by one or more computers and for respective reads of the obtained plurality of reads, a score corresponding to respective base calls of the respective reads that match the plurality of reference positions where the known variant sequence exists (330). For example, the classification engine 160 can determine a base call error rate based on the score for each single-cell read. For example, the classification engine can determine that any single-cell read having a score that satisfies a predetermined threshold has a sufficient base call quality and those below it have insufficient base call quality. Then, the base call error rate can be determined as a ration of the single-cell reads having, e.g., insufficient base call quality over the total number of single-cell reads. These values can be used as input to the classification algorithm.

The process 300 includes classifying, by one or more computers, the single cell as a tumor cell or normal cell based on an aggregation of the score determined for the respective reads of the obtained plurality of reads (340). For example, the classification engine 160 can generate a likelihood, based upon execution of equation (5) above, that a cell is a tumor cell or a normal cell. In some implementations, the classification engine 160 can determine whether the generated likelihood satisfies a predetermined threshold. If the classification engine 160 determines that the generated likelihood satisfies the predetermined threshold, then the classification engine can generate output data 184 indicating that the single cell is a tumor cell. Alternatively, if the classification engine 160 determines that the generated likelihood does not satisfy the predetermined threshold, the classification engine 160 can generate output data 184 indicating that the single cell is a normal cell.

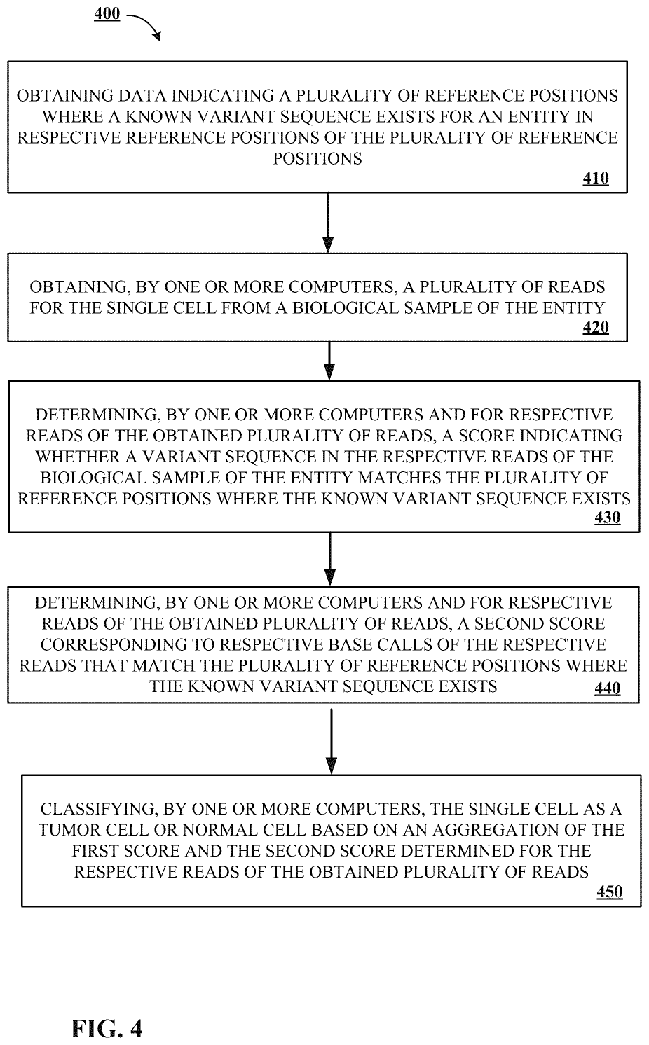

FIG. 4 is a flowchart of an example of a process 400 for performing classification of single cells as tumor or normal from single cell sequences. The process 400 may be performed by one or more electronic systems, for example, the system 100 of FIG. 1.

The process 400 includes obtaining data indicating a plurality of reference positions where a known variant sequence exists for an entity in respective reference positions of the plurality of reference positions (410). For example, functionality of the read alignment unit 136 obtaining data indicating a plurality of reference positions can include obtaining a reference sequence. In some example embodiments, a reference sequence is a sequence that includes one or more known non-variant reference sequences in the respective reference positions. In some example embodiments, a reference sequence is a sequence that includes one or more known variant sequences in respective reference positions and one or more known non-variant reference sequences in the respective reference positions. In some example, obtaining data indicating a plurality of reference positions includes obtaining the reference sequence.

The process 400 includes obtaining, by one or more computers, a plurality of reads for the single cell from a biological sample of the entity (420). For example, the functionality of the read alignment unit 136 can also include obtaining one or more reads such as RNA reads 112-1, 112-2, 112-n that were stored in memory 120 by the sequencing device 110, mapping the obtained reads 112-1, 112-2, 112-n to one or more reference sequence locations of a reference sequence, and then aligning the mapped reads 112-1, 112-2, 112-n to the reference sequence.

The process 400 includes determining, by one or more computers and for respective reads of the obtained plurality of reads, a score indicating whether a variant sequence in the respective reads of the biological sample of the entity matches the plurality of reference positions where the known variant sequence exists (430). For example, the classification engine 160 can use the first score to provide an indication of (i) a number of single-cell reads that support a known variant sequence 113 and (ii) a number of single-cell reads that support a known reference sequence 115. By way of example, the number of single-cell reads supporting a known variant sequence can be a sum of the number of single-cell reads that have a “1” as their score and the number of single-cell reads supporting a known reference can be a sum of the number of reads having a “0” as their score. These values can be used as input to the classification algorithm.

The process 400 includes determining, by one or more computers and for respective reads of the obtained plurality of reads, a second score corresponding to respective base calls of the respective reads that match the plurality of reference positions where the known variant sequence exists (440). For example, the classification engine 160 can determine a base call error rate based on the second score for each single-cell read. For example, the classification engine can determine that any single-cell read having a second score that satisfies a predetermined threshold has a sufficient base call quality and those below it have insufficient base call quality. Then, the base call error rate can be determined as a ration of the single-cell reads having, e.g., insufficient base call quality over the total number of single-cell reads. These values can be used as input to the classification algorithm.

The process 400 includes classifying, by one or more computers, the single cell as a tumor cell or normal cell based on an aggregation of the first score and the second score determined for the respective reads of the obtained plurality of reads (450). For example, the classification engine 160 can generate a likelihood, based upon execution of equation (5) above, that a cell is a tumor cell or a normal cell. In some implementations, the classification engine 160 can determine whether the generated likelihood satisfies a predetermined threshold. If the classification engine 160 determines that the generated likelihood satisfies the predetermined threshold, then the classification engine can generate output data 184 indicating that the single cell is a tumor cell. Alternatively, if the classification engine 160 determines that the generated likelihood does not satisfy the predetermined threshold, the classification engine 160 can generate output data 184 indicating that the single cell is a normal cell.

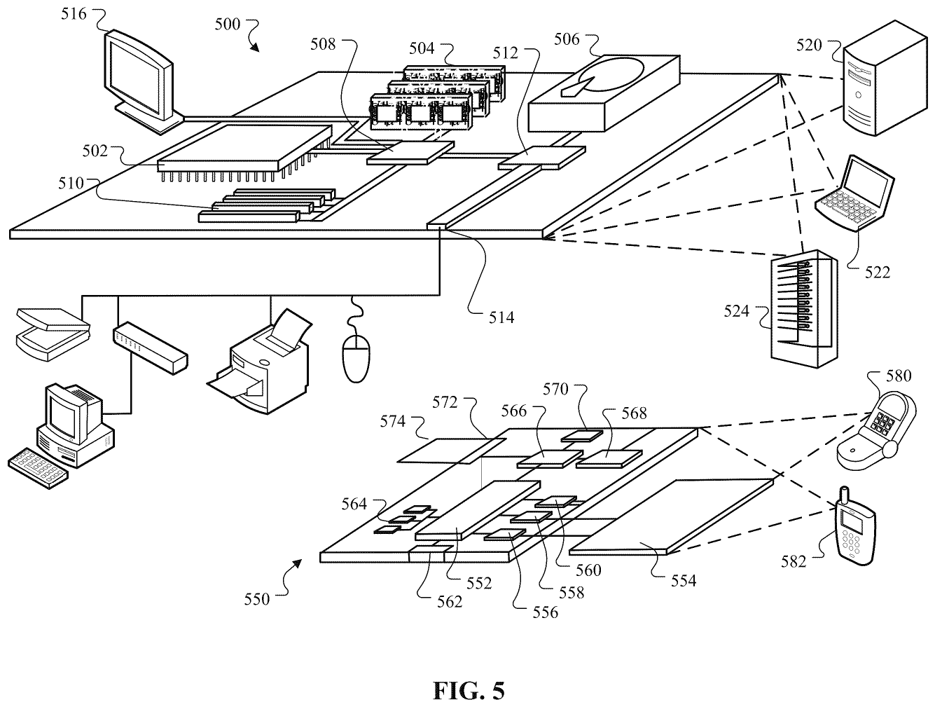

FIG. 5 is a block diagram of system components that can be used to implement a system for classification of single cells as tumor or normal from single cell sequences.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high speed controller 508 manages bandwidth-intensive operations for the computing device 500, while the low speed controller 512 manages lower bandwidth intensive operations. Such allocation of functions is exemplary only. In one implementation, the high-speed controller 508 is coupled to memory 504, display 516, e.g., through a graphics processor or accelerator, and to high-speed expansion ports 510, which can accept various expansion cards (not shown). In the implementation, low-speed controller 512 is coupled to storage device 506 and low-speed expansion port 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor 510 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provide in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. Such expansion memory 574 can provide extra storage space for device 550, or can also store applications or other information for device 550. Specifically, expansion memory 574 can include instructions to carry out or supplement the processes described above, and can include secure information also. Thus, for example, expansion memory 574 can be provide as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 580. It can also be implemented as part of a smartphone 582, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### OTHER EMBODIMENTS

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A method for classification of a single cell from a biological sample of an entity, the method comprising:
obtaining data indicating a plurality of reference positions where a known variant sequence exists for the entity in respective reference positions of the plurality of reference positions;
obtaining, by one or more computers, a plurality of reads for the single cell from the biological sample of the entity;
determining, by one or more computers and for respective reads of the obtained plurality of reads, a score indicating whether a variant sequence in the respective reads of the biological sample of the entity matches the plurality of reference positions where the known variant sequence exists; and
classifying, by one or more computers, the single cell as a tumor cell or normal cell based on an aggregation of the score determined for the respective reads of the obtained plurality of reads.

2. The method of claim 1, further comprising:
determining, by one or more computers and for respective reads of the obtained plurality of reads, a quality score corresponding to respective base calls of the respective reads corresponding to the known variant sequence, wherein the score indicating whether a known variant sequence of the biological sample of the entity is present in the respective reads includes the quality score.

3. The method of claim 1, further comprising:
obtaining a reference sequence, wherein:
the reference sequence is a sequence that includes one or more known variant sequences in the respective reference positions or one or more known non-variant reference sequences in the respective reference positions; and
the reference sequence is sequenced from a tissue sample obtained from the entity.

4. The method of claim 3, wherein obtaining data indicating a plurality of reference positions includes obtaining the reference sequence.

5. The method of claim 3, wherein the one or more known non-variant reference sequences include sequences that do not include one or more tumor-normal (TN) somatic variants.

6. The method of claim 3, wherein the one or more known variant sequences in the respective reference positions include one or more TN somatic variants.

7. The method of claim 1, wherein the single cell from the biological sample is isolated from a non-tumor sample from the entity.

8. The method of claim 1, wherein the single cell from the biological sample is isolated from a tumor sample from the entity.

9. A system for classification of a single cell from a biological sample of an entity, the system comprising:
one or more computers; and
one or more memory devices storing instructions that, when executed by the one or more computers, cause the one or more computers to perform operations, the operations comprising:
obtaining data indicating a plurality of reference positions where a known variant sequence exists for the entity in respective reference positions of the plurality of reference positions;
obtaining, by one or more computers, a plurality of reads for the single cell from the biological sample of the entity;
determining, by one or more computers and for respective reads of the obtained plurality of reads, a score indicating whether a variant sequence in the respective reads of the biological sample of the entity matches the plurality of reference positions where the known variant sequence exists; and
classifying, by one or more computers, the single cell as a tumor cell or normal cell based on an aggregation of the score determined for the respective reads of the obtained plurality of reads.

10. The system of claim 9, the operations comprising:
determining, by one or more computers and for respective reads of the obtained plurality of reads, a quality score corresponding to respective base calls of the respective reads corresponding to the known variant sequence, wherein the score indicating whether a known variant sequence of the biological sample of the entity is present in the respective reads includes the quality score.

11. The system of claim 9, the operations comprising:
obtaining a reference sequence, wherein:
the reference sequence is a sequence that includes one or more known variant sequences in the respective reference positions or one or more known non-variant reference sequences in the respective reference positions; and
the reference sequence is sequenced from a tissue sample obtained from the entity.

12. The operations of claim 11, wherein obtaining data indicating a plurality of reference positions includes obtaining the reference sequence.

13. The operations of claim 11, wherein the one or more known non-variant reference sequences include sequences that do not include one or more tumor-normal (TN) somatic variants.

14. The operations of claim 11, wherein the one or more known variant sequences in the respective reference positions include one or more TN somatic variants.

15. The operations of claim 9, wherein the single cell from the biological sample is isolated from a non-tumor sample from the entity.

16. The method of claim 1, wherein the single cell from the biological sample is isolated from a tumor sample from the entity.

17. One or more computer-readable storage media storing instructions that, when executed by the one or more computers, cause the one or more computers to perform operations for classification of a single cell from a biological sample of an entity, the operations comprising:
obtaining data indicating a plurality of reference positions where a known variant sequence exists for the entity in respective reference positions of the plurality of reference positions;
obtaining, by one or more computers, a plurality of reads for the single cell from the biological sample of the entity;
determining, by one or more computers and for respective reads of the obtained plurality of reads, a score indicating whether a variant sequence in the respective reads of the biological sample of the entity matches the plurality of reference positions where the known variant sequence exists; and
classifying, by one or more computers, the single cell as a tumor cell or normal cell based on an aggregation of the score determined for the respective reads of the obtained plurality of reads.

18. The computer-readable storage media of claim 17, the operations comprising:
determining, by one or more computers and for respective reads of the obtained plurality of reads, a quality score corresponding to respective base calls of the respective reads corresponding to the known variant sequence, wherein the score indicating whether a known variant sequence of the biological sample of the entity is present in the respective reads includes the quality score.

19. The computer-readable storage media of claim 17, the operations comprising:
obtaining a reference sequence, wherein:
the reference sequence is a sequence that includes one or more known variant sequences in the respective reference positions or one or more known non-variant reference sequences in the respective reference positions; and
the reference sequence is sequenced from a tissue sample obtained from the entity.

20. The computer-readable storage media of claim 17, wherein the single cell from the biological sample is isolated from a non-tumor sample from the entity.
